SEQUENCE LISTING (3 sequences)

SEQ ID NO: 1 - DNA, 3213 bp, Sesamum indicum L.
  source 1..3213: mol_type = genomic DNA
cagacaggtctatatttatgagacctcgtaaggcagaacgcaacgccactcaaatatttcaccaccaccaccaccagaacattcagaaacaagaaataaacacacacacactaataaaacagttcttgcgaaagaaggaaagcgcttccgcagaagtgctttcacgcgatttcttcttccaagttttcaggtaacgtgcccccttttctcttctcttctattctcttttctcataattcatgatcaatctttgagtattttggtgtttgtgtgtctcaagaaaaccgcatttttattttcttgcaatggtgtctttatttcctgtcgtttttttcagctattaatgttcttttgatgtagatgaggtttaatcgtatgttcttgagctgcattacctgatgattcatggatctgaggaatgtatgcgattttttatttttgttttattttttggtgggctttcccaagaagaatctatttggggttattcttgtgtggtttggtgcaaatctttggattttacgcagtattggtgtctggaccacatgattgtgtcatttatatttggattttgtctttatctttgtatgcatgtgggatgcaggaagaaaaaactgtggtaaatgtctttgaagagattgatttagcatatatacaaggttgcctgggcttcagttttgatgattttgatgtacattgtggagatttgatgggttgcatgtggctcaaatcttcttgtaagatttgttttttgtccaaaaaatttgggatttttccacttttattgaacagtagatcttttcctgtttcaaccacaaaagttatttcggtttgaagttttacatcatgatataattgtaataaatttcggttaggtccgtaaagaatcattaattacatcaattaatattgtttaatgtacaaaaagagggaatttatggtgatatctatgaagccatgctatgcctggctggaattccgtcgatgaaaaagacagattccggtgtgtggtagatttcactgttagtgaataccccacttcaaagaacggtgctgattcaactgctctagtcctcaggattttagtactacttgtttgctgtttggaacacatggctgaaaataaatgtctgcttttcgaccttggcgcttagagaatttactaccacatctcatttttagcatcccaacgatgatttctgctgtcagaatgaatgaattgactaagaagcaactcggttatttgagattgaattggttgtttgtgattgttgttgatttgtttttgtcgttatgatcttttgaggtattcgccatacaatgctgatactagtcgttgtgattttccggtatatgtatttgtgacgtatcgttctgtagtttggtaactaatagaatgcatgtggtggtaactaatagaatgcatgttgtagtaacaaatgcacattgtagattctcgtggatttttcgggtgttcgttaccagcacattgccgattctggtatgatttttgtcgtgttcattgtttagttgcctttcttggctgccactatttcattgagaatgtaggacgttgttcgatgcaaaagaacttttgccgactagaatgcaggtggcaatctggaatctcctattatgggaggaactactgtaattgggaggttttgattcagacaatctagtaacagtctagaagctactttgcctttaaatctcaatgaccttaaacgccatgatggagacatttgaatccatgttttgcaggtaaattgggggcggcttgacaaaatgggagccggaggacgcatgtctgatccaacaacgaaagacgaacaaaagaagaaccccctccaacgggtgccttacgcaaagcctccattcacactcggtgacatcaagaaggccattccaccacactgcttcgagagatccgtcagccgttcgttctcctatgtcgtttacgatctcgtcattgttttccttctctactacattgcgacttcttacttccatctgctgccatccccatactgctacctagcttggcccatttactgggctgtacaaggctgcgtttgcaccggaatctgggtcattgcccatgaatgtggccaccatgcattcagcgattaccagtggcttgacgacacagttggcctcatcctgcactctgccctgctcgtgccctatttctcatggaaatacagccaccgccgccaccactccaacactggatcccttgagcgtgacgaagtcttcgtcccaaagccaaaatccagagtctcgtggtactccaaatacttgaacaatccacttggcagagtcatcacacttgtggttactcttactctcggttggcctctatacttgctgtttaatgtctctggcaggccttacaaccgttttgcatgccactttgacccatatggtccaatatataatgaccgtgagagacttcaaatcttcatctccgatgctggtataattgctgctgtatgtgtgctttatcgtgttgctttggtcaaagggttggcttggctggtatgtgtttatggggtaccgttactcattgtcaacggtttctttgttttgatcacattccttcagcacactcacccttcgttgccgcactatgattcttccgagtgggactggctaaggggagctcttgcaactgtcgacagagattatggggtgctaaataaggtgttccataacatcacagatacgcacgtgactcaccaccttttctcaacgatgccacattaccatgcaatggaggcaactaaggcaatcaagcccatactgggccagtattatcagtttgatggaaccccgttttacaaggcgatgtggagggaggcaaaggaatgtctgtatgtcgagccagacgagagtactccagacaagggtgtattctggtacaagaacaagttctgaagccgaataacatgtggttagtgaaaatggcgtcttcttattttgtcctatggagatggaggaacatcatcatgttttctttttcttcttataagatgcgtcctttgttagtgtattctctgcatgtaataaaataaacttctacccgaaaccttgtctgtgctggtcggattctagttctgcaataaattgtcaagtttagtgatggcttcaggtcttttgttagagattttcttccatcctactacataataatgtgctaatattcctgct

SEQ ID NO: 2 - DNA, 3213 bp, Sesamum indicum L.
  source 1..3213: mol_type = genomic DNA
cagacaggtctatatttatgagacctcgtaaggcagaacgcaacgccactcaaatatttcaccaccaccaccaccagaacattcagaaacaagaaataaacacacacacactaataaaacagttcttgcgaaagaaggaaagcgcttccgcagaagtgctttcacgcgatttcttcttccaagttttcaggtaacgtgcccccttttctcttctcttctattctcttttctcataattcatgatcaatctttgagtattttggtgtttgtgtgtctcaagaaaaccgcatttttattttcttgcaatggtgtctttatttcctgtcgtttttttcagctattaatgttcttttgatgtagatgaggtttaatcgtatgttcttgagctgcattacctgatgattcatggatctgaggaatgtatgcgattttttatttttgttttattttttggtgggctttcccaagaagaatctatttggggttattcttgtgtggtttggtgcaaatctttggattttacgcagtattggtgtctggaccacatgattgtgtcatttatatttggattttgtctttatctttgtatgcatgtgggatgcaggaagaaaaaactgtggtaaatgtctttgaagagattgatttagcatatatacaaggttgcctgggcttcagttttgatgattttgatgtacattgtggagatttgatgggttgcatgtggctcaaatcttcttgtaagatttgttttttgtccaaaaaatttgggatttttccacttttattgaacagtagatcttttcctgtttcaaccacaaaagttatttcggtttgaagttttacatcatgatataattgtaataaatttcggttaggtccgtaaagaatcattaattacatcaattaatattgtttaatgtacaaaaagagggaatttatggtgatatctatgaagccatgctatgcctggctggaattccgtcgatgaaaaagacagattccggtgtgtggtagatttcactgttagtgaataccccacttcaaagaacggtgctgattcaactgctctagtcctcaggattttagtactacttgtttgctgtttggaacacatggctgaaaataaatgtctgcttttcgaccttggcgcttagagaatttactaccacatctcatttttagcatcccaacgatgatttctgctgtcagaatgaatgaattgactaagaagcaactcggttatttgagattgaattggttgtttgtgattgttgttgatttgtttttgtcgttatgatcttttgaggtattcgccatacaatgctgatactagtcgttgtgattttccggtatatgtatttgtgacgtatcgttctgtagtttggtaactaatagaatgcatgtggtggtaactaatagaatgcatgttgtagtaacaaatgcacattgtagattctcgtggatttttcgggtgttcgttaccagcacattgccgattctggtatgatttttgtcgtgttcattgtttagttgcctttcttggctgccactatttcattgagaatgtaggacgttgttcgatgcaaaagaacttttgccgactagaatgcaggtggcaatctggaatctcctattatgggaggaactactgtaattgggaggttttgattcagacaatctagtaacagtctagaagctactttgcctttaaatctcaatgaccttaaacgccatgatggagacatttgaatccatgttttgcaggtaaattgggggcggcttgacaaaatgggagccggaggacgcatgtctgatccaacaacgaaagacgaacaaaagaagaaccccctccaacgggtgccttacgcaaagcctccattcacactcggtgacatcaagaaggccattccaccacactgcttcgagagatccgtcagccgttcgttctcctatgtcgtttacgatctcgtcattgttttccttctctactacattgcgacttcttacttccatctgctgccatccccatactgctacctagcttggcccatttactgggctgtacaaggctgcgtttgcaccggaatctgggtcattgcccatgaatgtggccaccatgcattcagcgattaccagtggcttgacgacacagttggcctcatcctgcactctgccctgctcgtgccctatttctcatggaaatacagccaccgccgccaccactccaacactggatcccttgagcgtgacgaagtcttcgtcccaaagccaaaatccagagtctcgtggtactccaaatacttgaacaatccacttggcagagtcatcacacttgtggttactcttactctcggttggcctctatacttgctgtttaatgtctctggcaggccttacaaccgttttgcatgccactttgacccatatggtccaatatataatgaccgtgagagacttcaaatcttcatctccgatgctggtataattgctgctgtatgtgtgctttatcgtgttgctttggtcaaagggttggcttggctggtatgtgtttatggggtaccgttactcattgtcaacggtttccttgttttgatcacattccttcagcacactcacccttcgttgccgcactatgattcttccgagtgggactggctaaggggagctcttgcaactgtcgacagagattatggggtgctaaataaggtgttccataacatcacagatacgcacgtgactcaccaccttttctcaacgatgccacattaccatgcaatggaggcaactaaggcaatcaagcccatactgggccagtattatcagtttgatggaaccccgttttacaaggcgatgtggagggaggcaaaggaatgtctgtatgtcgagccagacgagagtactccagacaagggtgtattctggtacaagaacaagttctgaagccgaataacatgtggttagtgaaaatggcgtcttcttattttgtcctatggagatggaggaacatcatcatgttttctttttcttcttataagatgcgtcctttgttagtgtattctctgcatgtaataaaataaacttctacccgaaaccttgtctgtgctggtcggattctagttctgcaataaattgtcaagtttagtgatggcttcaggtcttttgttagagattttcttccatcctactacataataatgtgctaatattcctgct

SEQ ID NO: 3 - DNA, 1398 bp, Sesamum indicum L.
  source 1..1398: mol_type = other DNA
atgagacctcgtaaggcagaacgcaacgccactcaaatatttcaccaccaccaccaccagaacattcagaaacaagaaataaacacacacacactaataaaacagttcttgcgaaagaaggaaagcgcttccgcagaagtgctttcacgcgatttcttcttccaagttttcagattctcgtggatttttcgggtgttcgttaccagcacattgccgattctggtaaattgggggcggcttgacaaaatgggagccggaggacgcatgtctgatccaacaacgaaagacgaacaaaagaagaaccccctccaacgggtgccttacgcaaagcctccattcacactcggtgacatcaagaaggccattccaccacactgcttcgagagatccgtcagccgttcgttctcctatgtcgtttacgatctcgtcattgttttccttctctactacattgcgacttcttacttccatctgctgccatccccatactgctacctagcttggcccatttactgggctgtacaaggctgcgtttgcaccggaatctgggtcattgcccatgaatgtggccaccatgcattcagcgattaccagtggcttgacgacacagttggcctcatcctgcactctgccctgctcgtgccctatttctcatggaaatacagccaccgccgccaccactccaacactggatcccttgagcgtgacgaagtcttcgtcccaaagccaaaatccagagtctcgtggtactccaaatacttgaacaatccacttggcagagtcatcacacttgtggttactcttactctcggttggcctctatacttgctgtttaatgtctctggcaggccttacaaccgttttgcatgccactttgacccatatggtccaatatataatgaccgtgagagacttcaaatcttcatctccgatgctggtataattgctgctgtatgtgtgctttatcgtgttgctttggtcaaagggttggcttggctggtatgtgtttatggggtaccgttactcattgtcaacggtttccttgttttgatcacattccttcagcacactcacccttcgttgccgcactatgattcttccgagtgggactggctaaggggagctcttgcaactgtcgacagagattatggggtgctaaataaggtgttccataacatcacagatacgcacgtgactcaccaccttttctcaacgatgccacattaccatgcaatggaggcaactaaggcaatcaagcccatactgggccagtattatcagtttgatggaaccccgttttacaaggcgatgtggagggaggcaaaggaatgtctgtatgtcgagccagacgagagtactccagacaagggtgtattctggtacaagaacaagttctga

(19) United States
(12) Patent Application Publication
(10) Pub. No.: US 2025/0122585 A1
(43) Pub. Date: Apr. 17, 2025

(54) SESAME HIGH OLEIC ACID CONTENT GENE SIFAD2-1 AND SNP MARKER THEREOF

(71) Applicant: HENAN SESAME RESEARCH CENTER, HENAN ACADEMY OF AGRICULTURAL SCIENCES, Zhengzhou (CN)

(72) Inventors: HONGMEI MIAO, Zhengzhou (CN); HAIYANG ZHANG, Zhengzhou (CN); QIN MA, Zhengzhou (CN); HENGCHUN CAO, Zhengzhou (CN); MING JU, Zhengzhou (CN); SHUO WANG, Zhengzhou (CN); CHUN LI, Zhengzhou (CN); YINGHUI DUAN, Zhengzhou (CN); ZHANYOU ZHANG, Zhengzhou (CN)

(21) Appl. No.: 18/983,211

(22) Filed: Dec. 16, 2024

Related U.S. Application Data
(63) Continuation of application No. PCT/CN2024/075023, filed on Jan. 31, 2024.

(51) Int. Cl. C12Q 1/6895; C12N 9/02; C12Q 1/6806; C12Q 1/6809; C12Q 1/686

(52) U.S. Cl. CPC C12Q 1/6895; C12N 9/0071; C12Q 1/6806; C12Q 1/6809; C12Q 1/686; C12Q 2600/13; C12Y 114/19

(57) ABSTRACT

A gene SiFAD2-1 controlling the oleic acid content trait in sesame and its SNP marker SiSNPFAD2-1 are provided. This gene is located on chromosome 4 of sesame and belongs to an incomplete dominant control gene; compared with a wild-type allele Sifad2-1. The high oleic acid mutant gene SiFAD2-1 has a 100% explanation value for a mutant trait of sesame high oleic acid mutant HO995 (i.e., this gene controls a high oleic acid phenotype). The present application can provide a certain theoretical basis for studying the regulation mechanism of high oil acid in sesame and other crops, and also provide a material basis and genetic resources for developing molecular assisted breeding technology for sesame, breeding new varieties with high oleic acid in sesame and even other crops.

### CROSS-REFERENCE TO RELATED APPLICATIONS

This application is a continuation of International Application No. PCT/CN2024/075023, filed on Jan. 31, 2024, which claims priority to Chinese Patent Application No. 202211721838.0 filed on Dec. 30, 2022, both of which are hereby incorporated by reference in their entireties.

### TECHNICAL FIELD

The present disclosure belongs to the field of sesame molecular genetic breeding technologies, and in particular, to a gene, SiFAD2-1 controlling oleic acid content trait in sesame, and a SNP marker SiSNPFAD2-1 thereof.

### SEQUENCE LISTING

The present application contains a sequence listing which was filed electronically in XML format and is hereby incorporated by reference in its entirety. Besides, the XML copy is created on Dec. 12, 2024, is named “SESAME HIGH OLEIC ACID CONTENT GENE SIFAD2-1 AND SNP MARKER THEREOF-Sequence Listing” and is 11,424 bytes in sizes.

### BACKGROUND

Sesame (Sesamum indicum L., 2n=26) is a specific oilseed crop with high quality. Sesame seeds are enriched of unsaturated fatty acids, proteins, dietary fiber, and antioxidants and are widely utilized in food processing, nutritional health products, and the pharmaceutical industry. Studies have shown that oleic acid (C18:1) and linoleic acid (C18:2) are the primary fatty acids in sesame seeds, accounting for more than 80% of the total fatty acid content, and offer health benefits such as lowering blood lipids and preventing coronary heart disease and cholesterol-related issues.

As oleic acid content is one of the most critical evaluation indicators for the quality of oilseed crops, the exploration of oleic acid content regulation genes through gene breeding technology holds the significant technical guidance value for breeding new crop varieties.

### SUMMARY

On the basis of the sesame mutant material HO995 with high oleic acid content, the inventors identified and cloned a sesame gene, SiFAD2-1 (encoding FAD2 protein) related to high oleic acid content for establishing the technical foundation for breeding new varieties with high oleic acid content in sesame and other crops.

The technical solution of the present application is as following.

The sesame high oleic acid content gene SiFAD2-1, located on the 4th chromosome of sesame, is an incomplete dominant control gene (compared to the normal phenotype allele, the mutated site is located at the 2584th base);
compared with the wild-type allele Sifad2-1, the high oleic acid content mutant gene SiFAD2-1 has a 100% explanation ratio for the mutant trait in the sesame high oleic acid content mutant HO995 (i.e., this gene control presents the high oleic acid content phenotype);
   this gene has a length of 3213 bp, contains 3 exons and 2 introns, and its base sequence is shown in SEQ ID No. 1, as following:

CAGACAGGTCTATATTTATGAGACCTCGTAAGGCAGAACGCAACGCCACTCAAA
TATTTCACCACCACCACCACCAGAACATTCAGAAACAAGAAATAAACACACACAC
ACTAATAAAACAGTTCTTGCGAAAGAAGGAAAGCGCTTCCGCAGAAGTGCTTTCA
CGCGATTTCTTCTTCCAAGTTTTCAGGTAACGTGCCCCCTTTTCTCTTCTCTTCTATT
CTCTTTTCTCATAATTCATGATCAATCTTTGAGTATTTTGGTGTTTGTGTGTCTCAAG
AAAACCGCATTTTTATTTTCTTGCAATGGTGTCTTTATTTCCTGTCGTTTTTTTCAGC
TATTAATGTTCTTTTGATGTAGATGAGGTTTAATCGTATGTTCTTGAGCTGCATTACC
TGATGATTCATGGATCTGAGGAATGTATGCGATTTTTTATTTTTGTTTTATTTTTTGGT
GGGCTTTCCCAAGAAGAATCTATTTGGGGTTATTCTTGTGTGGTTTGGTGCAAATCT
TTGGATTTTACGCAGTATTGGTGTCTGGACCACATGATTGTGTCATTTATATTTGGAT
TTTGTCTTTATCTTTGTATGCATGTGGGATGCAGGAAGAAAAAACTGTGGTAAATGT
CTTTGAAGAGATTGATTTAGCATATATACAAGGTTGCCTGGGCTTCAGTTTTGATGA
TTTTGATGTACATTGTGGAGATTTGATGGGTTGCATGTGGCTCAAATCTTCTTGTAA
GATTTGTTTTTTGTCCAAAAAATTTGGGATTTTTCCACTTTTATTGAACAGTAGATCT
TTTCCTGTTTCAACCACAAAAGTTATTTCGGTTTGAAGTTTTACATCATGATATAATT
GTAATAAATTTCGGTTAGGTCCGTAAAGAATCATTAATTACATCAATTAATATTGTTT
AATGTACAAAAAGAGGGAATTTATGGTGATATCTATGAAGCCATGCTATGCCTGGCT
GGAATTCCGTCGATGAAAAAGACAGATTCCGGTGTGTGGTAGATTTCACTGTTAGT
GAATACCCCACTTCAAAGAACGGTGCTGATTCAACTGCTCTAGTCCTCAGGATTTT
AGTACTACTTGTTTGCTGTTTGGAACACATGGCTGAAAATAAATGTCTGCTTTTCGA
CCTTGGCGCTTAGAGAATTTACTACCACATCTCATTTTTAGCATCCCAACGATGATT
TCTGCTGTCAGAATGAATGAATTGACTAAGAAGCAACTCGGTTATTTGAGATTGAA
TTGGTTGTTTGTGATTGTTGTTGATTTGTTTTTGTCGTTATGATCTTTTGAGGTATTC
GCCATACAATGCTGATACTAGTCGTTGTGATTTTCCGGTATATGTATTTGTGACGTAT
CGTTCTGTAGTTTGGTAACTAATAGAATGCATGTGGTGGTAACTAATAGAATGCATG
TTGTAGTAACAAATGCACATTGTAGATTCTCGTGGATTTTTCGGGTGTTCGTTACCA
GCACATTGCCGATTCTGGTATGATTTTTGTCGTGTTCATTGTTTAGTTGCCTTTCTTG
GCTGCCACTATTTCATTGAGAATGTAGGACGTTGTTCGATGCAAAAGAACTTTTGC
CGACTAGAATGCAGGTGGCAATCTGGAATCTCCTATTATGGGAGGAACTACTGTAA
TTGGGAGGTTTTGATTCAGACAATCTAGTAACAGTCTAGAAGCTACTTTGCCTTTA
AATCTCAATGACCTTAAACGCCATGATGGAGACATTTGAATCCATGTTTTGCAGGTA
AATTGGGGGCGGCTTGACAAAATGGGAGCCGGAGGACGCATGTCTGATCCAACAA
CGAAAGACGAACAAAAGAAGAACCCCCTCCAACGGGTGCCTTACGCAAAGCCTC
CATTCACACTCGGTGACATCAAGAAGGCCATTCCACCACACTGCTTCGAGAGATCC
GTCAGCCGTTCGTTCTCCTATGTCGTTTACGATCTCGTCATTGTTTTCCTTCTCTACT
ACATTGCGACTTCTTACTTCCATCTGCTGCCATCCCCATACTGCTACCTAGCTTGGC
CCATTTACTGGGCTGTACAAGGCTGCGTTTGCACCGGAATCTGGGTCATTGCCCAT
GAATGTGGCCACCATGCATTCAGCGATTACCAGTGGCTTGACGACACAGTTGGCCT
CATCCTGCACTCTGCCCTGCTCGTGCCCTATTTCTCATGGAAATACAGCCACCGCCG
CCACCACTCCAACACTGGATCCCTTGAGCGTGACGAAGTCTTCGTCCCAAAGCCA
AAATCCAGAGTCTCGTGGTACTCCAAATACTTGAACAATCCACTTGGCAGAGTCAT
CACACTTGTGGTTACTCTTACTCTCGGTTGGCCTCTATACTTGCTGTTTAATGTCTCT
GGCAGGCCTTACAACCGTTTTGCATGCCACTTTGACCCATATGGTCCAATATATAAT
GACCGTGAGAGACTTCAAATCTTCATCTCCGATGCTGGTATAATTGCTGCTGTATGT
GTGCTTTATCGTGTTGCTTTGGTCAAAGGGTTGGCTTGGCTGGTATGTGTTTATGGG
GTACCGTTACTCATTGTCAACGGTTTCTTTGTTTTGATCACATTCCTTCAGCACACT
CACCCTTCGTTGCCGCACTATGATTCTTCCGAGTGGGACTGGCTAAGGGGAGCTCT
TGCAACTGTCGACAGAGATTATGGGGTGCTAAATAAGGTGTTCCATAACATCACAG
ATACGCACGTGACTCACCACCTTTTCTCAACGATGCCACATTACCATGCAATGGAG
GCAACTAAGGCAATCAAGCCCATACTGGGCCAGTATTATCAGTTTGATGGAACCCC
GTTTTACAAGGCGATGTGGAGGGAGGCAAAGGAATGTCTGTATGTCGAGCCAGAC
GAGAGTACTCCAGACAAGGGTGTATTCTGGTACAAGAACAAGTTCTGAAGCCGAA
TAACATGTGGTTAGTGAAAATGGCGTCTTCTTATTTTGTCCTATGGAGATGGAGGAA
CATCATCATGTTTTCTTTTTCTTCTTATAAGATGCGTCCTTTGTTAGTGTATTCTCTGC
ATGTAATAAAATAAACTTCTACCCGAAACCTTGTCTGTGCTGGTCGGATTCTAGTTC
TGCAATAAATTGTCAAGTTTAGTGATGGCTTCAGGTCTTTTGTTAGAGATTTTCTTC
CATCCTACTACATAATAATGTGCTAATATTCCTGCT.

An allele Sifad2-1 of the sesame high oleic acid content gene SiFAD2-1, a plant phenotype of this allele is a normal phenotype (or a phenotype with a normal oleic acid content; i.e., the 2584th base in the normal phenotype is C, while the 2584th base in the high oleic acid content mutant is T), and its base sequence is shown in SEQ ID No. 2, as following:

CAGACAGGTCTATATTTATGAGACCTCGTAAGGCAGAACGCAACGCCACTCAAA
TATTTCACCACCACCACCACCAGAACATTCAGAAACAAGAAATAAACACACACAC
ACTAATAAAACAGTTCTTGCGAAAGAAGGAAAGCGCTTCCGCAGAAGTGCTTTCA
CGCGATTTCTTCTTCCAAGTTTTCAGGTAACGTGCCCCCTTTTCTCTTCTCTTCTATT
CTCTTTTCTCATAATTCATGATCAATCTTTGAGTATTTTGGTGTTTGTGTGTCTCAAG
AAAACCGCATTTTTATTTTCTTGCAATGGTGTCTTTATTTCCTGTCGTTTTTTTCAGC
TATTAATGTTCTTTTGATGTAGATGAGGTTTAATCGTATGTTCTTGAGCTGCATTACC
TGATGATTCATGGATCTGAGGAATGTATGCGATTTTTTATTTTTGTTTTATTTTTTGGT
GGGCTTTCCCAAGAAGAATCTATTTGGGGTTATTCTTGTGTGGTTTGGTGCAAATCT
TTGGATTTTACGCAGTATTGGTGTCTGGACCACATGATTGTGTCATTTATATTTGGAT
TTTGTCTTTATCTTTGTATGCATGTGGGATGCAGGAAGAAAAAACTGTGGTAAATGT
CTTTGAAGAGATTGATTTAGCATATATACAAGGTTGCCTGGGCTTCAGTTTTGATGA
TTTTGATGTACATTGTGGAGATTTGATGGGTTGCATGTGGCTCAAATCTTCTTGTAA
GATTTGTTTTTTGTCCAAAAAATTTGGGATTTTTCCACTTTTATTGAACAGTAGATCT
TTTCCTGTTTCAACCACAAAAGTTATTTCGGTTTGAAGTTTTACATCATGATATAATT
GTAATAAATTTCGGTTAGGTCCGTAAAGAATCATTAATTACATCAATTAATATTGTTT
AATGTACAAAAAGAGGGAATTTATGGTGATATCTATGAAGCCATGCTATGCCTGGCT
GGAATTCCGTCGATGAAAAAGACAGATTCCGGTGTGTGGTAGATTTCACTGTTAGT
GAATACCCCACTTCAAAGAACGGTGCTGATTCAACTGCTCTAGTCCTCAGGATTTT
AGTACTACTTGTTTGCTGTTTGGAACACATGGCTGAAAATAAATGTCTGCTTTTCGA
CCTTGGCGCTTAGAGAATTTACTACCACATCTCATTTTTAGCATCCCAACGATGATT
TCTGCTGTCAGAATGAATGAATTGACTAAGAAGCAACTCGGTTATTTGAGATTGAA
TTGGTTGTTTGTGATTGTTGTTGATTTGTTTTTGTCGTTATGATCTTTTGAGGTATTC
GCCATACAATGCTGATACTAGTCGTTGTGATTTTCCGGTATATGTATTTGTGACGTAT
CGTTCTGTAGTTTGGTAACTAATAGAATGCATGTGGTGGTAACTAATAGAATGCATG
TTGTAGTAACAAATGCACATTGTAGATTCTCGTGGATTTTTCGGGTGTTCGTTACCA
GCACATTGCCGATTCTGGTATGATTTTTGTCGTGTTCATTGTTTAGTTGCCTTTCTTG
GCTGCCACTATTTCATTGAGAATGTAGGACGTTGTTCGATGCAAAAGAACTTTTGC
CGACTAGAATGCAGGTGGCAATCTGGAATCTCCTATTATGGGAGGAACTACTGTAA
TTGGGAGGTTTTGATTCAGACAATCTAGTAACAGTCTAGAAGCTACTTTGCCTTTA
AATCTCAATGACCTTAAACGCCATGATGGAGACATTTGAATCCATGTTTTGCAGGTA
AATTGGGGGCGGCTTGACAAAATGGGAGCCGGAGGACGCATGTCTGATCCAACAA
CGAAAGACGAACAAAAGAAGAACCCCCTCCAACGGGTGCCTTACGCAAAGCCTC
CATTCACACTCGGTGACATCAAGAAGGCCATTCCACCACACTGCTTCGAGAGATCC
GTCAGCCGTTCGTTCTCCTATGTCGTTTACGATCTCGTCATTGTTTTCCTTCTCTACT
ACATTGCGACTTCTTACTTCCATCTGCTGCCATCCCCATACTGCTACCTAGCTTGGC
CCATTTACTGGGCTGTACAAGGCTGCGTTTGCACCGGAATCTGGGTCATTGCCCAT
GAATGTGGCCACCATGCATTCAGCGATTACCAGTGGCTTGACGACACAGTTGGCCT
CATCCTGCACTCTGCCCTGCTCGTGCCCTATTTCTCATGGAAATACAGCCACCGCCG
CCACCACTCCAACACTGGATCCCTTGAGCGTGACGAAGTCTTCGTCCCAAAGCCA
AAATCCAGAGTCTCGTGGTACTCCAAATACTTGAACAATCCACTTGGCAGAGTCAT
CACACTTGTGGTTACTCTTACTCTCGGTTGGCCTCTATACTTGCTGTTTAATGTCTCT
GGCAGGCCTTACAACCGTTTTGCATGCCACTTTGACCCATATGGTCCAATATATAAT
GACCGTGAGAGACTTCAAATCTTCATCTCCGATGCTGGTATAATTGCTGCTGTATGT
GTGCTTTATCGTGTTGCTTTGGTCAAAGGGTTGGCTTGGCTGGTATGTGTTTATGGG
GTACCGTTACTCATTGTCAACGGTTTCCTTGTTTTGATCACATTCCTTCAGCACACT
CACCCTTCGTTGCCGCACTATGATTCTTCCGAGTGGGACTGGCTAAGGGGAGCTCT
TGCAACTGTCGACAGAGATTATGGGGTGCTAAATAAGGTGTTCCATAACATCACAG
ATACGCACGTGACTCACCACCTTTTCTCAACGATGCCACATTACCATGCAATGGAG
GCAACTAAGGCAATCAAGCCCATACTGGGCCAGTATTATCAGTTTGATGGAACCCC
GTTTTACAAGGCGATGTGGAGGGAGGCAAAGGAATGTCTGTATGTCGAGCCAGAC
GAGAGTACTCCAGACAAGGGTGTATTCTGGTACAAGAACAAGTTCTGAAGCCGAA
TAACATGTGGTTAGTGAAAATGGCGTCTTCTTATTTTGTCCTATGGAGATGGAGGAA
CATCATCATGTTTTCTTTTTCTTCTTATAAGATGCGTCCTTTGTTAGTGTATTCTCTGC
ATGTAATAAAATAAACTTCTACCCGAAACCTTGTCTGTGCTGGTCGGATTCTAGTTC
TGCAATAAATTGTCAAGTTTAGTGATGGCTTCAGGTCTTTTGTTAGAGATTTTCTTC
CATCCTACTACATAATAATGTGCTAATATTCCTGCT.

The cDNA corresponding to the allele Sifad2-1 of the sesame high oleic acid content gene SiFAD2-1 (i.e. the cDNA corresponding to the normal oleic acid content phenotype) encodes 465 amino acids; its base sequence (1398 bp in length) is shown in SEQ ID NO. 3, as following:

ATGAGACCTCGTAAGGCAGAACGCAACGCCACTCAAATATTTCACCACCACCAC
CACCAGAACATTCAGAAACAAGAAATAAACACACACACACTAATAAAACAGTTCT
TGCGAAAGAAGGAAAGCGCTTCCGCAGAAGTGCTTTCACGCGATTTCTTCTTCCA
AGTTTTCAGATTCTCGTGGATTTTTCGGGTGTTCGTTACCAGCACATTGCCGATTCT
GGTAAATTGGGGGCGGCTTGACAAAATGGGAGCCGGAGGACGCATGTCTGATCCA
ACAACGAAAGACGAACAAAAGAAGAACCCCCTCCAACGGGTGCCTTACGCAAAG
CCTCCATTCACACTCGGTGACATCAAGAAGGCCATTCCACCACACTGCTTCGAGAG
ATCCGTCAGCCGTTCGTTCTCCTATGTCGTTTACGATCTCGTCATTGTTTTCCTTCTC
TACTACATTGCGACTTCTTACTTCCATCTGCTGCCATCCCCATACTGCTACCTAGCTT
GGCCCATTTACTGGGCTGTACAAGGCTGCGTTTGCACCGGAATCTGGGTCATTGCC
CATGAATGTGGCCACCATGCATTCAGCGATTACCAGTGGCTTGACGACACAGTTGG
CCTCATCCTGCACTCTGCCCTGCTCGTGCCCTATTTCTCATGGAAATACAGCCACCG
CCGCCACCACTCCAACACTGGATCCCTTGAGCGTGACGAAGTCTTCGTCCCAAAG
CCAAAATCCAGAGTCTCGTGGTACTCCAAATACTTGAACAATCCACTTGGCAGAGT
CATCACACTTGTGGTTACTCTTACTCTCGGTTGGCCTCTATACTTGCTGTTTAATGTC
TCTGGCAGGCCTTACAACCGTTTTGCATGCCACTTTGACCCATATGGTCCAATATAT
AATGACCGTGAGAGACTTCAAATCTTCATCTCCGATGCTGGTATAATTGCTGCTGTA
TGTGTGCTTTATCGTGTTGCTTTGGTCAAAGGGTTGGCTTGGCTGGTATGTGTTTAT
GGGGTACCGTTACTCATTGTCAACGGTTTCCTTGTTTTGATCACATTCCTTCAGCAC
ACTCACCCTTCGTTGCCGCACTATGATTCTTCCGAGTGGGACTGGCTAAGGGGAGC
TCTTGCAACTGTCGACAGAGATTATGGGGTGCTAAATAAGGTGTTCCATAACATCA
CAGATACGCACGTGACTCACCACCTTTTCTCAACGATGCCACATTACCATGCAATG
GAGGCAACTAAGGCAATCAAGCCCATACTGGGCCAGTATTATCAGTTTGATGGAAC
CCCGTTTTACAAGGCGATGTGGAGGGAGGCAAAGGAATGTCTGTATGTCGAGCCA
GACGAGAGTACTCCAGACAAGGGTGTATTCTGGTACAAGAACAAGTTCTGA.

The specific encoded amino acid sequence (465 amino acids, with the 348th amino acid L) is:

MRPRKAERNATQIFHHHHHQNIQKQEINTHTLIKQFLRKKESASAEVLSRDFFFQVF
RFSWIFRVFVTSTLPILVNWGRLDKMGAGGRMSDPTTKDEQKKNPLQRVPYAKPPFT
LGDIKKAIPPHCFERSVSRSFSYVVYDLVIVFLLYYIATSYFHLLPSPYCYLAWPIYWAV
QGCVCTGIWVIAHECGHHAFSDYQWLDDTVGLILHSALLVPYFSWKYSHRRHHSNT
GSLERDEVFVPKPKSRVSWYSKYLNNPLGRVITLVVTLTLGWPLYLLFNVSGRPYNRF
ACHFDPYGPIYNDRERLQIFISDAGIIAAVCVLYRVALVKGLAWLVCVYGVPLLIVNGF
LVLITFLQHTHPSLPHYDSSEWDWLRGALATVDRDYGVLNKVFHNITDTHVTHHLFS
TMPHYHAMEATKAIKPILGQYYQFDGTPFYKAMWREAKECLYVEPDESTPDKGVFW
YKNKF.

The cDNA corresponding to the sesame high oleic acid gene SiFAD2-1 differs from the cDNA corresponding to the allele Sifad2-1 of the normal oleic acid phenotype in that the 1042nd nucleotide in the cDNA sequence of the normal allele is C, and the 1042nd nucleotide of the cDNA sequence corresponding to the mutated high oleic acid gene SiFAD2-1 is T;
correspondingly, the encoded amino acid sequence (with the 348th amino acid F) is as follows:

MRPRKAERNATQIFHHHHHQNIQKQEINTHTLIKQFLRKKESASAEVLSRDFFFQVF
RFSWIFRVFVTSTLPILVNWGRLDKMGAGGRMSDPTTKDEQKKNPLQRVPYAKPPFT
LGDIKKAIPPHCFERSVSRSFSYVVYDLVIVFLLYYIATSYFHLLPSPYCYLAWPIYWAV
QGCVCTGIWVIAHECGHHAFSDYQWLDDTVGLILHSALLVPYFSWKYSHRRHHSNT
GSLERDEVFVPKPKSRVSWYSKYLNNPLGRVITLVVTLTLGWPLYLLFNVSGRPYNRF
ACHFDPYGPIYNDRERLQIFISDAGIIAAVCVLYRVALVKGLAWLVCVYGVPLLIVNGF
FVLITFLQHTHPSLPHYDSSEWDWLRGALATVDRDYGVLNKVFHNITDTHVTHHLFS
TMPHYHAMEATKAIKPILGQYYQFDGTPFYKAMWREAKECLYVEPDESTPDKGVFW
YKNKF.

A primer pair for PCR amplification to obtain the sesame high oleic acid content gene SiFAD2-1 or its allele Sifad2-1 is as follows:

a positive Primer HO1 Primer F:
5′-TATGAGACCTCGTAAGGCAG-3′;
a reverse primer HO1 Primer R:
5′-GTAGGATGGAAGAAAATCTC-3′.

A PCR amplification method for obtaining the sesame high oleic acid content gene SiFAD2-1 or its allele Sifad2-1 using the primer pair for PCR amplification, includes the following steps:
(1) extracting genomic DNA from the high oleic acid content mutant HO995 or from a sesame germplasm sample with normal oleic acid content;
(2) using the genomic DNA extracted in step (1) as a template, perform PCR amplification with the primer pair;
when using the genomic DNA of the high oleic acid mutant HO995 as the template, the sesame high oleic acid content gene SiFAD2-1 can be obtained after amplification;
when using the genomic DNA of the sesame sample with normal oleic acid content as the template, the allele Sifad2-1 can be obtained after amplification.

A primer pair for HRM PCR (high resolution melting curve PCR) detection of the sesame high oleic acid content gene SiFAD2-1 and its allele Sifad2-1 is named as a SNP marker SiSNPfad2-1, and includes the following:

HSHO1-F sequence:
5′-AAGGGTTGGCTTGGCTGGTA-3′; (Tm: 59.7° C.)
HSHO1-R sequence:
5′-CGGCAACGAAGGGTGAGTG-3′ (Tm: 59.8° C.);

the amplified product has a length of 104 bp;
the sequence of the amplification product is as follows (the 60th locus is the different site):

AAGGGTTGGCTTGGCTGGTATGTGTTTATGGGGTACCGTTACTCATTGT
CAACGGTTTCTTTGTTTTGATCACATTCCTTCAGCACACTCACCCTTCG
TTGCCG.

A method for detecting and determining the sesame oleic acid content phenotype using the primer pair (SNP marker SiSNPfad2-1) includes the following steps:
(1) extracting genomic DNA from the sesame sample to be tested;
(2) using the genomic DNA extracted in step (1) as a template, perform HRM PCR amplification with the SNP marker SiSNPfad2-1;
(3) comparing the HRM PCR peak results with a control and determining whether the sample has the high oleic acid content phenotype or the normal oleic acid phenotype;

Based on the HRM PCR design and the C/T mutation site, the genotype of the sample is determined as follows:
if a peak line appears after amplification, the sample is a C/T genotype, indicating the sample is heterozygous (with moderate oleic acid content), where the oleic acid content is between that of the mutant and the normal sample;
if a smooth line appears after amplification, the sample is either CC genotype or TT genotype, and further steps are required to determine whether it is a homozygous with high oleic acid phenotype or normal oleic acid phenotype.

In order to distinguish the CC from TT homozygous genotype, an equal amount of normal oleic acid content control DNA template (i.e. CC genotype) can be added to the homozygous sample's template DNA, and a second HRM PCR is performed:
if a peak line appears after amplification, the sample is the TT genotype (inconsistent with the control genotype), indicating that the sample contains the mutant SNP site with homozygous mutant phenotype (high oleic acid mutant TT);
if a smooth line persists after PCR amplification, the mixed sample is a homozygous CC genotype, indicating that the sample does not contain the T mutation SNP site with a homozygous normal phenotype (CC genotype with normal oleic acid content).

In recent years, the applicant (Henan Sesame Research Center, Henan Academy of Agricultural Sciences) has created a high oleic acid mutant, HO995 from the widely cultivated sesame variety Yuzhi 11 via EMS mutagenesis technology, and cultivated a new high oleic acid sesame variety, Yuzhi HO995, with an oleic acid content of 80%. The test results indicated that the oleic acid content in the mutant HO995 accounted for over 80% of the total fatty acids, and is significantly higher than that of common sesame germplasm and varieties, in which the oleic acid content in the total fats ranges from 35 to 45%.

In order to determine the gene sequence differences between the high oleic acid mutant HO995 and the wild type, and obtain the gene controlling the high oleic acid content trait in sesame, the inventors carried out the genetic analysis related to high oleic acid traits with the high oleic acid sesame mutant HO995 and the conducted F2 and F2:3 populations from the HO995*Yuzhi 11. Related studies revealed that the high oleic acid trait in the HO995 mutant is incompletely dominant and controlled by a single gene. Furthermore, using a mixed pool association analysis of sesame hybrid populations and the information of the high-quality sesame fine genome map, the inventors successfully cloned and identified the target gene, SiFAD2-1 regulating the high oleic acid trait in HO995 and developed a gene marker (SNP) corresponding to sesame high oleic acid content.

Overall, main technical procedures and advantages of the present application are reflected in the following aspects:
(1) during locating the high oleic acid gene in the new sesame variety HO995, a sesame high oleic acid gene SiFAD2-1 was cloned and a corresponding SNP marker SiSNPfad2-1 was developed; at the same time, a HRM PCR identification method was provided to determine whether a germplasm accession contains this gene and gene marker site; this method can conveniently and quickly determine whether the test sample is FAD2-1 phenotype with high oleic acid content or a hybrid, thereby providing a reference for new varieties breeding;
(2) the high oleic acid content trait and gene involved in the present application are of great significance for studying the regulation mechanism of sesame oil biosynthesis. They lay a certain technical and theoretical foundation for promoting molecular breeding and high-quality new variety selection in sesame, and thus have important technical significance for accelerating the process of sesame molecular assisted breeding;
(3) a detection method based on SNP marker provided by the present disclosure is a mature technology, the detection results are highly stable, which has important technical significance for improving the efficiency of sesame breeding and enhancing the level of sesame genetic breeding research technology.

In summary, the SiFAD2-1 gene provided in the present application has a 100% explanation ratio for the high oleic acid content trait in sesame. Through in-depth research on the gene SiFAD2-1 and its allele Sifad2-1, as well as a combination of specific SNP molecular marker SiSNPPAD2-1 and a corresponding detection method, a theoretical basis can be provided for studying the regulation and development mechanism of high oil acid trait in sesame and other crops. At the same time, it also provides material basis and genetic resources for developing molecular assisted breeding technology in sesame and breeding new varieties with high oleic acid in sesame and even other crops. Therefore, the application has good scientific research value and economic application value.

### BRIEF DESCRIPTION OF DRAWINGS

FIG. 1 illustrates the statistical result of the oleic acid content phenotype in the high oleic acid mutant HO995 and the wild type, and their hybrid F2 generation. The figure shows the distribution of the ratio of oleic acid C18:1 to the total fatty acids in seeds from HO995 (P1), wild type Yuzhi 11 (P2), and their hybrid F2 individuals (represented by black bars).

FIG. 2 shows the distribution of SNP-index values of the sesame high oleic acid and normal oleic acid bulked sample pools. In the figure, BSA1 and BSA2 represent two high oleic acid bulked pools; while BSA3 and BSA4 represent the two low oleic acid bulked pools. P1 refers to the high oleic acid parent HO995, and P2 refers to the normal oleic acid content parent Yuzhi 11. The black line indicates the fitted SNP-index.

FIG. 3 shows a structural comparison between the high oleic acid gene SiFAD2-1 and its allele Sifad2-1; in the colorful view: the blue box represents gene exons, the black horizontal line represents gene introns, and the green box represents 5′ and 3′ non-coding regions. The target SNP site in red shows a mutation from C2584 to T2584, and the amino acid Phe348 in red is mutated from Leu348.

FIG. 4 shows the HRM PCR results of the primer pair in different genotype samples for the SNP marker SiSNPfad2-1 associated with the sesame high oleic acid gene. The figure illustrates the amplification results of 72 samples;
in the colorful view: the red line represents the heterozygous C/T genotype (with oleic acid content falling between the high and the normal oleic acid content); while the blue line represents the homozygous CC or TT genotype (with either high oleic acid content or normal oleic acid content phenotype).

FIG. 5 shows the results of the second HRM PCR analysis in different genotype samples using the primer pair for the gene SNP marker SiSNPfad2-1 associated with sesame high oleic acid content, as described in the present disclosure. This figure presents the second amplification results of 24 homozygous samples added with equal amounts of control DNA. In the colorful view: the red line represents the peak in homozygous TT genotype sample mixed with CC control DNA, indicating the sample is high oleic acid phenotype (TT genotype); the blue line represents the flat line in CC genotype samples mixed with control DNA after amplification, indicating that the sample is normal phenotype (CC genotype) with normal oleic acid content.

FIG. 6 shows partial results of sample amplification using the primer pair for the sesame high oleic acid gene SNP marker SiSNPPAD2-1 in the present disclosure.

This figure contains 72 samples. In the colorful view: the red bands represent 20 heterozygous samples and 4 samples of mixed parents P1 and P2 DNA, respectively; the blue bands represent 22 samples with high oleic acid content (TT genotype) and 2 individual plantlets from the high oleic acid parent (P1-01, P1-02); 22 samples with normal oleic acid content (CC genotype) and 2 individual plantlets from the normal oleic acid parent (P2-01, P2-02).

### DESCRIPTION OF EMBODIMENTS

Below is a further explanation of the present application in conjunction with the provided Examples. Before introducing the specific Examples, a brief introduction and explanation of certain experimental backgrounds relevant to the following Examples are presented.

### Sesame Materials:

Yuzhi 11, a commonly used and widely cultivated sesame variety, can be obtained through public channels;
The sesame high oleic acid mutant HO995 was screened by the applicant (Sesame Research Center of Henan Academy of Agricultural Sciences) from Yuzhi 11 mutant populations created by artificial EMS mutagenesis technology. Based on this discovery, a new high oleic sesame variety, Yuzhi HO995, has been applied. This variety was officially ratified by Henan Crop Seed Management Station on Apr. 15, 2022, and a Henan Province Crop New Variety Identification Certificate (No.: Yupinjian Sesame 2022013) has been granted. The main traits of Yuzhi HO995 include normal growth and development and normal fat and protein content in the seeds, while the oleic acid content reaches more than 80% of total fatty acids, and significantly higher than the parental control (40%).

### Example 1

As mentioned above, based on the preliminary screening of the high oleic acid mutant HO995, during further cultivating new variety, a detailed localization research and analysis of the gene controlling the high oleic acid content trait in sesame were conducted. The specific research process is briefly introduced as follows.

### (I) Genetic Background Analysis of High Oil Acid Trait

From 2018 to 2020, the inventors chosen the high oleic acid mutant HO995 (with an oleic acid content exceeding 80%) and Yuzhi 11 (wt, normal oleic acid content, 40%) and performed hybrid combination (as detailed in Table 1). The fatty acid composition and oleic acid content traits of F1, F2, BC1, and BC2 offspring were investigated and statistically analyzed. The oleic acid content was measured using the near-infrared spectroscopy (NIRS) method established by Yuan Qingli et al. (2021).

In favor of the subsequent genetic analysis, in the statistical process:
samples with oleic acid content below 45% were classified as the normal wild-type (wt),
those with oleic acid content above 70% were classified as high oleic acid mutant type (HO995),
those with oleic acid content between 45-70% were classified as intermediate heterozygous types.

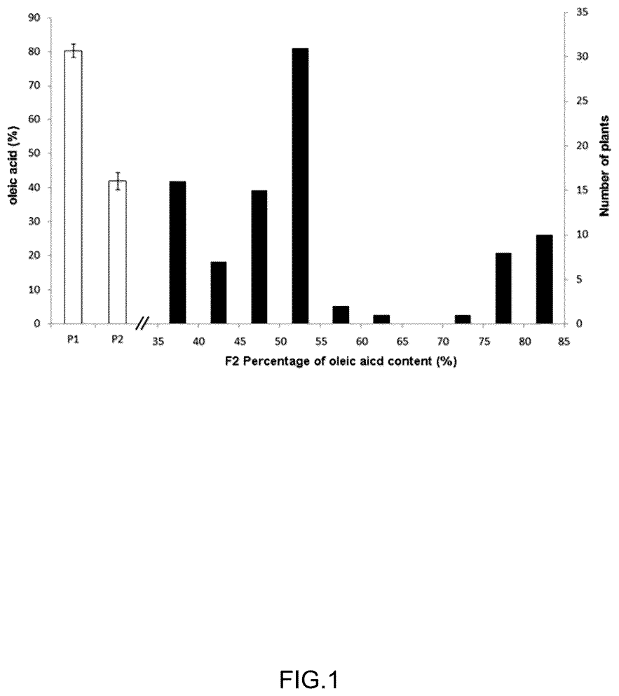

The specific hybrid combination and statistical results of the oleic acid content phenotypes are shown in Table 1 and FIG. 1.

TABLE 1
Information of sesame germplasm materials and phenotypic statistics of oleic acid content traits

| | | Phenotypic statistics of oleic acid | | | Number of individual plants in | | | |
|---|---|---|---|---|---|---|---|---|
| | | content F1 (plant number) | | | F2 population (plants) | | | |
| | | High | Moderate | Normal | High | Moderate | Normal | |
| | | oleic acid | oleic acid | oleic acid | oleic acid | oleic acid | oleic acid | |
| Hybrid combination | | content | content | content | content | content | content | |
| Parent 1 | Parent 2 | (>70%) | (45-70%) | (<40%) | (>70%) | (45-70%) | (<40%) | X² |
| HO995 (High | Yuzhi 11 (wt) | / | 10 | / | 31 | 70 | 35 | 0.35 |
| oleic acid) | | | | | | | | |
| Yuzhi 11 (wt) | HO995 (High | / | 10 | / | 29 | 73 | 48 | 4.92 |
| | oleic acid) | | | | | | | |

in the table, X²_{(0.05, 2)} = 5.99

Continued Table 1 Configuration of sesame germplasm materials and phenotypic statistics of oleic acid content traits

| | | Phenotypic statistics of oleic acid | | | |
|---|---|---|---|---|---|
| | | content in BC population | | | |
| | | High | Moderate | Normal | |
| | | oleic acid | oleic acid | oleic acid | |
| Hybrid combination | | content | content | content | Expected |
| Parent 1 | Parent 2 | (>70%) | (45-70%) | (<40%) | ratio |
| (HO995 * Yuzhi 11) F₁ | HO995 | 31 | 24 | 0 | 1:1 |
| | (High oleic acid) | | | | |
| (HO995 * Yuzhi 11) F₁ | Yuzhi 11 (wt) | 0 | 78 | 103 | 1:1 |
| (Yuzhi 11 * HO995) F₁ | HO995 | 77 | 91 | 0 | 1:1 |
| | (High oleic acid) | | | | |
| (Yuzhi 11 * HO995) F₁ | Yuzhi 11 (wt) | 0 | 48 | 47 | 1:1 |

The chi-square test results shown in the table above indicated that the ratio of high oleic acid:moderate oleic acid:normal oleic acid plants in F2 population accords with the expected 1:2:1, and the segregation ratio in the backcross population accords with 1:1. This result suggests that the inheritance of the oleic acid content trait in the high oleic acid mutant HO995 is in accordance with the Mendelian inheritance pattern, and is controlled by a single gene. Furthermore, based on F1 phenotype, it can be concluded that this gene exhibits incomplete dominance.

### (II) BSA Sequencing of Sesame High Oleic Acid and Normal Phenotypic Parents

Based on the aforementioned results, in 2021, the inventors further conducted phenotypic investigations of the F2-3 population of both forward and reverse crosses derived from the high oleic acid mutant HO995 and Yuzhi 11 (normal, wt).

In the investigation, the F2-3 population consisted of 1,402 individual plantlets. For each plantlet, 2-3 pieces of young leaves were collected and stored at −80° C. for future use. After the seeds were harvested from each plantlet, the oleic acid content was measured individually.

In addition, 30 samples with high oleic acid content and 30 samples with normal oleic acid content were randomly chosen from two groups of F2:3 populations, and DNA was extracted from the leaf samples to construct BSA pools. At the same time, DNA of parents was extracted for subsequent genome resequencing analysis.

### (III) Localization of Sesame High Oleic Acid Content Gene SiFAD2-1

On the basis the work in step (II), the inventors further localized the high oleic acid regulation gene in sesame. The specific work and process are briefly described as follows.

### (1) Re-Sequencing of Genomes of Parents and BSA

Firstly, for the eight groups of samples, involved in the two couples of parents and the four BSA pools mentioned above, DNA was extracted from each group using the improved CTAB method described by Wei Libin et al. (2008) (Sesame DNA and RNA Simultaneous Extraction Method, 2008, Molecular Plant Breeding). The genome re-sequencing of the BSA pools was performed using Illumina sequencing method, with a coverage of ≥30*.

Next, with the Yuzhi 11 genome data as a sesame reference genome, sequencing data from each line were aligned and assembled using BWA (Burrows Wheeler Aligner) software. Then, SNPs and InDels were detected and filtered using GATK (The Genome Analysis Toolkit). Combined with the above population data, the SNP-index values were calculated using the BSA analysis method.

Finally, the SNP-index screening method was applied, and the SNP-index and InDel-index difference values for the two groups of high oleic acid BSA and normal oleic acid BSA mixed pools were calculated, respectively, with P1 and P2 as reference parents. For the calculation, a completely identical SNP/InDel index is set to 0, and a completely different SNP/InDel index is set to 1.

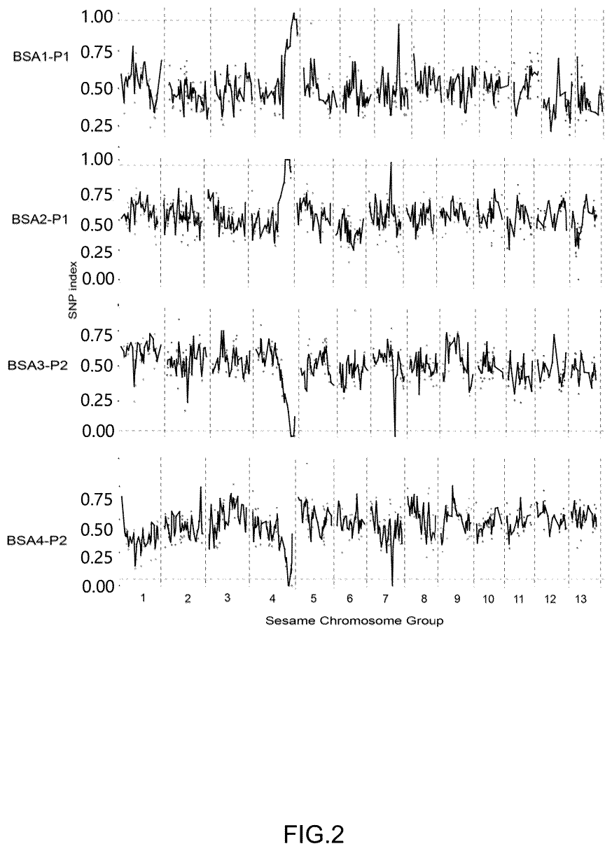

Based on this work, according to the SNP-index method and the correlation threshold, the variant regions on each chromosome closely associated with sesame oleic acid content trait are determined. A summary and statistical results are shown in FIG. 2.

Furthermore, after the results of four BSA groups were combined and analyzed, two variant regions on chromosome 4, specifically between 23669381-25859581 bp and 25862779-26124505 bp respectively, were identified to be closely associated with the sesame oleic acid content trait.

Further statistical analysis of variant position and mutation type within these regions revealed that there is only one non-synonymous SNP mutation at position 23786388. This SNP had an SNP-index value of 1, compared to the normal phenotype parent (as shown in Table 2 below). The results suggest that this marker has an explanation value of 1 for the phenotype variation. The analysis results showed that the SNP23786388 site is located within Sindi_1057100 gene, which encodes the A-12 fatty acid desaturase FAD2 enzyme.

TABLE 2
Information on variant loci significantly associated
with the oleic acid content trait

| | | | Reference | | SNP- |
|---|---|---|---|---|---|
| Chromosome | | Marker | genome | | index |
| No. | Marker No. | location | base | Variant | value |
| SiChr. 4 | SNP23786388 | 23786388 | C | T | 1 |

Based on the above analysis results, further validation of the SNP loci was conducted using population samples from the HO995*Yuzhi 11 and natural germplasm accessions. The results further confirmed that the SNP23786388 loci is closely linked to the high oleic acid trait.

### Example 2

On the basis of a preliminary localization in Example 1, the located sesame high oleic acid gene was further cloned and sequenced. A specific process is briefly introduced as follows.

Based on the variant locus obtained in Example 1 above, the SNP23786388 locus was analyzed and determined as a target SNP locus using the Yuzhi 11 genome data, and the gene Sindi_1057100 where the SNP is located was determined as the target gene.

Sequence analysis revealed that the gene was annotated as a [symbol] -12 fatty acid desaturase gene in the sesame genome (Yuzhi 11), hence the target gene was named SiFAD2-1 gene.

Based on the above analysis work and Yuzhi 11 genome data, a primer pair for PCR amplification was designed to clone the target gene: high oleic acid gene SiFAD2-1 or its allele Sifad2-1; the specific sequence of the designed primers is:

a positive Primer HO1 Primer F
5′-TATGAGACCTCGTAAGGCAG-3′;
a reverse primer HO1 Primer R:
5′-GTAGGATGGAAGAAAATCTC-3′.

Subsequently, taking the DNA extracted from the high oleic acid germplasm HO995 or the sesame sample with normal oleic acid content (Yuzhi 11) as the template (the extraction method refers to Example 1), PCR amplification was performed with the above primer pair and high-fidelity enzyme PrimeSTAR Max DNA Polymerase (Takara product);
during PCR amplification, the reference setting for 20 μL reaction system is as follows:
DNA, 1 μL;
dNTP Mix, 0.4 μL (10 mM each);
2*Phanta Max Buffer, 10 μL;
Phanta Max Super-Fidelity DNA Polymerase, 0.4 μL;
EvaGreen, 1 μL;
Primer F, 0.4 μL;
Primer R, 0.4 μL;
ddH₂O, 6.4 μL;
when PCR amplification (PTC-100 thermal cycler (a product of MJ Research company), the reference design for a reaction procedure is: denaturation at 98° C. for 10 seconds, renaturation at 55° C. for 5 seconds, extension at 72° C. for 20 seconds, and circulation for 30 times; finally, extension at 72° C. for 5 minutes; an amplification product can be directly subjected to a subsequent electrophoresis analysis or stored at 4° C. for future use.

Electrophoresis detection was performed on the amplification product, and the amplification product was recovered for sequencing analysis (completed by Tianjin Gene Chip Biotechnology Co., Ltd.). The results indicate that:
the full-length genome sequence of the target gene (high oleic acid phenotype) SiFAD2-1 is 3123 bp, containing 3 exons and 2 introns, and the sequence is shown as SEQ ID No. 1;
the gene sequence of the normal gene (normal oleic acid content phenotype) Sifad2-1 is shown as SEQ ID No. 2.

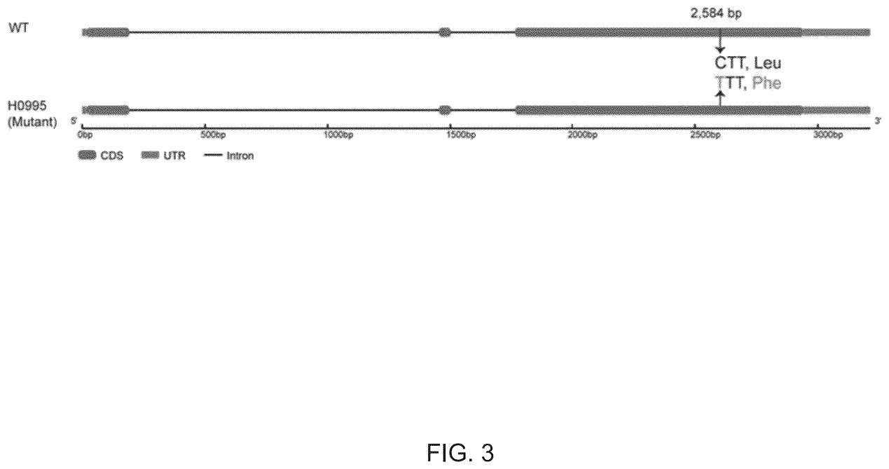

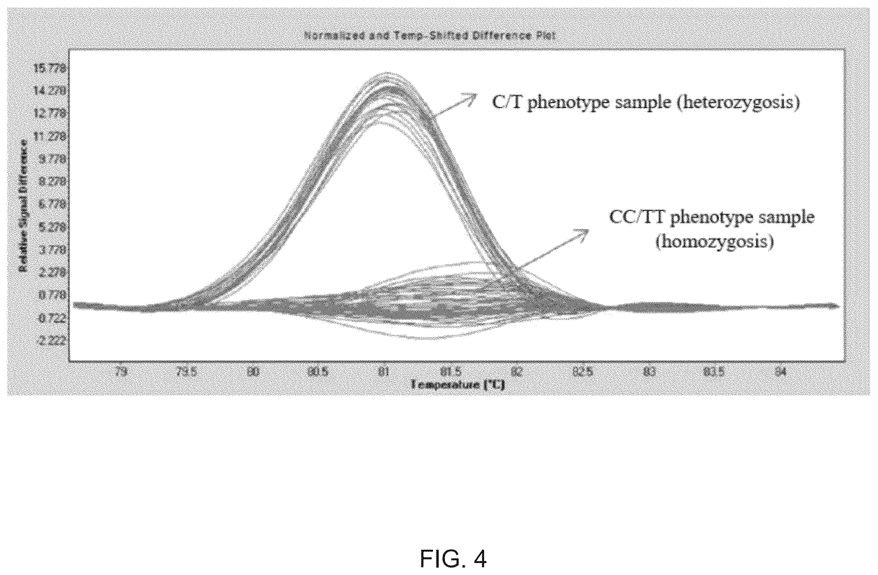

A sequence comparison between the high oleic acid gene SiFAD2-1 and its normal phenotype allele Sifad2-1 was conducted, and the schematic diagram is shown in FIG. 3. Specific analysis shows that:
in the genomes of sesame germplasm accessions with the high oleic acid phenotype and the normal phenotype, the only difference between SiFAD2-1 and the allele Sifad2-1 is in a C/T mutation at position 2584th of the gene sequence. When the 2584th base cytosine (C), is mutated to thymine (T), the 348th amino acid in the encoded protein is changed from leucine (Leu, L) to phenylalanine (Phe, F), thereby the oleic acid content phenotype is changed from normal to high level.

### Example 3

Based on the results of Example 2, in order to facilitate the application of the high oleic acid gene SiFAD2-1 in breeding work, the inventors designed a primer pair for PCR detection using HRM PCR technology, according to the characteristics of the corresponding SNP site. The primer pair was named SiSNPFAD2-1. At the same time, three genotype samples and phenotype data of offspring lines were chosen for preliminary validation. A brief introduction to the specific process is as follows.

Firstly, a designed PCR primer pair (SiSNPFAD2-1) is as follows:

HSHO1-F sequence:
5′-AAGGGTTGGCTTGGCTGGTA-3′; (Tm: 59.7° C.)
HSHO1-R sequence:
5′-CGGCAACGAAGGGTGAGTG-3′ (Tm: 59.8° C.).

Subsequently, based on the principles of HRM PCR technology, the specific detection process is as follows:
using the genomic DNA extracted from a test sesame sample as a template, perform PCR amplification with the primer pair (SiSNPFAD2-1);
during PCR amplification, the reference setting for a 15 μL reaction system is as follows:
Template DNA (50 ng/μL), 1.0 μL;
2*Phanta Max Buffer, 7.5 μL;
Phanta Max Super-Fidelity DNA Polymerase, 0.3 μL;
dNTP Mix, 0.3 μL;
Forward Primer (10 μM), 0.3 μL;
Reverse Primer (10 μM), 0.3 μL;
EvaGreen, 0.75 μL;
adding ultrapure water to 15 μL;
during a PCR process (Roche 480 PCR thermal cycler, Roche, Germany), a general PCR reaction procedure is: 95° C., 5 minutes; 95° C., 10 seconds; 63° C., 10 seconds; 72° C., 10 seconds; 40 cycles;
a reaction program of HRM is: 95° C., 1 minute; 40° C., 1 minute; 65° C., 1 second.

Finally, based on a comparison of the HRM PCR peak results with a control, the test sample is determined to be a high oleic acid gene genotype or a normal oleic acid genotype.

Considering that the SNP marker site in the present application belongs to a C/T mutation, the specific genotype determination process is as follows:
if a peak is observed in the sample, the sample has a C/T genotype, indicating that the sample is heterozygous (with moderate oleic acid content), where the oleic acid content is between that of the mutant and the normal sample;
if a smooth line is observed, the sample has either a CC or TT genotype, indicating that the sample is either a homozygous high oleic acid genotype or a homozygous normal genotype; in order to further distinguish whether the sample is homozygous CC or TT, an equal amount of normal oleic acid content control DNA template (i.e., CC genotype) is added to the homozygous sample DNA template, and a second HRM PCR is performed:
if a peak appears in the amplification result, the sample is the TT genotype (inconsistent with the control genotype), indicating that the sample contains the SNP mutation site and is a homozygous mutant (high oleic acid mutant TT genotype);
if a smooth line persists after PCR amplification, the mixed sample should be homozygous CC, indicating that the sample does not contain the T mutation SNP site, and should be a homozygous normal genotype (CC genotype with normal oleic acid content).

As mentioned earlier, during the detection process, three genotype samples and phenotype data of offspring lines were selected for the preliminary validation. Specifically:
200 F2 individual plants were randomly chosen from the F2 population of HO995 (high oleic acid) and Yuzhi 11 (normal oleic acid content). Young leaves from these individual plants were collected (pre-stored at −80° C.) for genomic DNA extraction, and the seeds of these 200 individual F2 plantlets were harvested. Under appropriate conditions, the seeds from these 200 individual plantlets were planted in rows. After harvesting, the fatty acids and oleic acid content of the seeds were tested. Ten individual plants per row were investigated. According to F2-3 phenotype investigation results, individual homozygous F2 plantlets with high oleic acid content, homozygous F2 plantlets with normal oleic acid content, and heterozygous F2 plantlets with moderate oleic acid content were chosen as samples for marker reliability testing. During testing and validating, genomic DNA from the three groups of F2 individual plantlets mentioned above were extracted, appropriate amounts of DNA from the three samples were taken, and equal amounts of homozygous positive control (Yuzhi 11) DNA were added. HRM PCR amplification verification was then performed according to the aforementioned procedure.

Some of the results are shown in FIG. 4. In the colorful view, the red line in FIG. 4 represents the heterozygous C/T phenotype (oleic acid content between the high and the normal phenotype), while the blue line represents the homozygous CC or TT genotype (with high oleic acid content or normal phenotype).

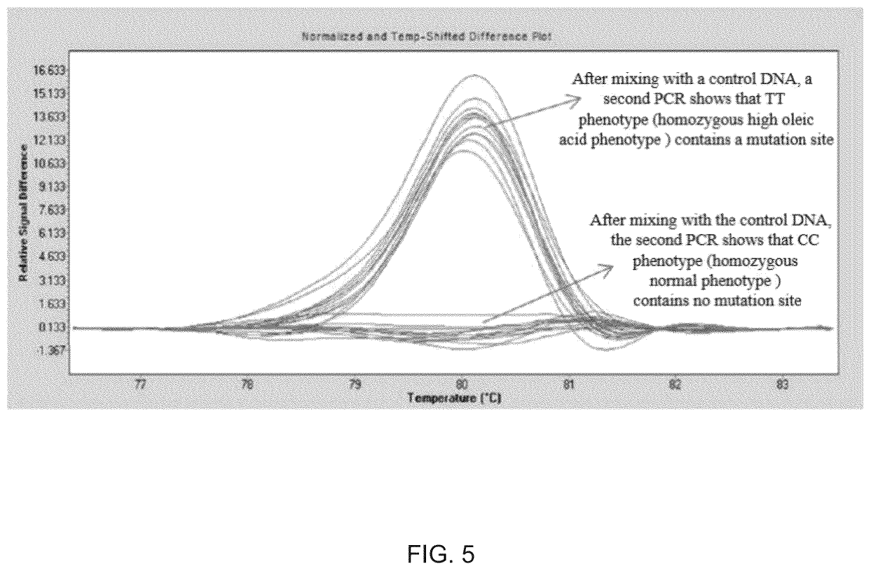

Furthermore, an equal amount of the normal oleic acid content control DNA template (i.e., CC type) was added to the homozygous sample DNA for HRM PCR. Some of the results are shown in FIG. 5. In the colorful view, the red line in FIG. 5 represents a peak line for the homozygous TT genotype sample mixed with the CC genotype control DNA after amplification, indicating that the sample has a high oleic acid phenotype. The blue line represents a flat line of the CC genotype sample mixed with the control DNA after amplification, indicating that the sample has a normal phenotype.

In the above results:
FIG. 4 shows 24 heterozygous plant samples (in red), which displayed a blue C/T peak band (i.e., containing both SNP alleles C/T), indicating a heterozygous genotype. 24 homozygous samples with high oleic acid content and 24 homozygous samples with normal content all exhibited blue bands (i.e., containing homozygous sites);
FIG. 5 presents 10 chosen homozygous plantlets with high oleic acid content, 10 homozygous plantlets with normal phenotype, 2 parent plantlets with high oleic acid content, and 2 parent plantlets with normal oleic acid content, which were mixed with the control DNA and subjected to HRM PCR. The results showed that, in the 10 homozygous samples with high oleic acid content and 2 high oleic acid parent plantlets, a peak band (red) appeared after amplification with mixed samples, indicating that the samples contain the SNP allele locus TT. After mixing with the control DNA, the 10 homozygous plantlets with normal phenotype and 2 normal parent plantlets still exhibit a bule flat line after amplification (i.e., containing a homozygous CC site).

Based on the relevant phenotype identification control results, the consistency rate between PCR and phenotype identification results was 100%. This indicates that the SNP marker and detection method are accurate and reliable.

### Example 4

Based on the results of Example 3, to further verify the screening and identification efficiency of the provided primer pair, the inventors conducted additional experimental verification. The specific process is briefly introduced as follows.

Firstly, 100 samples were randomly chosen from the hybrid F2 offspring of HO995 (high oleic acid) and Yuzhi 11 (normal oleic acid content) for planting. Young leaves of each F2 plantlets were collected (stored at −80° C. in advance) for genomic DNA extraction, and seeds from 100 F2 individual plants were harvested. These seeds were then sown in row manner. After harvested, the oleic acid content of the seeds was measured, and 20 individual plantlets were investigated in each row.

At the same time, 100 homozygous germplasm samples were randomly chosen from the germplasm library and planted in rows. During this period, young leaves were collected (stored at −80° C. in advance). After harvesting, the oleic acid content of the seeds was investigated. For each germplasm accession, 20 individual plantlets were examined.

Subsequently, genomic DNA was extracted from the 100 F2 individual plantlets and 100 representative sesame germplasm accessions. HRM PCR detection was then carried out with the SNP primer pair designed in Example 3 to evaluate the reliability of the SNP marker.

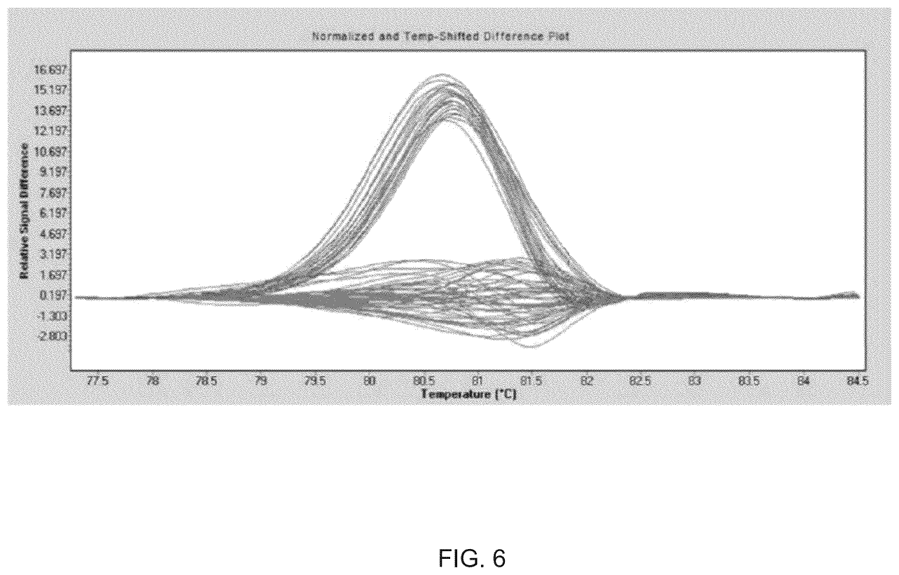

A part of the PCR results is shown in FIG. 6. The figure contains 72 samples, of which 48 samples represented by blue bands, contain 22 high oleic acid content samples (TT genotype) from the F2 population, including 3517-217, 3517-222, 3517-196, 3517-216, 3518-061, 3518-125, 3518-139, 3518-161, 3519-033, 3519-043, 3519-123, 3519-177, 3520-128, 3520-006, 3520-196, 3520-212, 3521-066, 3521-236, 3521-020, 3521-182, 3522-151, and 3522-155, and two parental plantlets with high oleic acid content (P1-01 P1-02), 22 samples with normal oleic acid content (CC genotype) from the F2 populations, including 3517-176, 3517-091, 3517-224, 3517-067, 3518-177, 3518-123, 3518-152, 3518-025, 3519-155, 3519-208, 3519-195, 3519-095, 3520-200, 3520-214, 3520-018, 3520-004, 3521-171, 3521-190, 3521-029, 3521-175, 3522-193, 3522-080, and 2 parental plantlets (P2-01, P1-02) with normal oleic acid content. (Sample no. such as “3517-217”, “3517-222”, “3517-196”, and others do not have special meanings).

The 24 samples represented by red bands, contain 22 heterozygous samples (C/T genotype) with oil content between the two parents, including 3517-174, 3517-214, 3517-161, 3517-191, 3518-028, 3518-176, 3518-053, 3518-026, 3519-007, 3519-197, 3519-137, 3519-168, 3520-113, 3520-032, 3520-143, 3520-111, 3521-125, 3521-001, 3522-088, and 3522-152, and 2 parental P1 and P2 DNA mixed samples (M-01, M-02).

In the tested materials, the phenotypic detection results indicated that the oleic acid content of the samples in the red bands (C/T genotype) ranged from 45 to 65%, which falls between the two parents. This result meets the expected target.

Furthermore, the blue band samples were mixed with the control DNA and subjected to a second amplification. The results indicated that red peak bands appeared in all 24 detected high oleic acid samples. The oleic acid content of the samples was also greater than 70%. Based on these results, it can be concluded that the detection accuracy of the primer pair in Example 3 is 100%. The results demonstrate that the SNP marker and the corresponding detection method described in the present application are accurate and reliable.

In summary, we believe that the SiSNPPAD2-1 marker represents the SNP loci marker of the high oleic acid gene in sesame. It can be used to predict the oleic acid content phenotype and origins of sesame samples, and is useful for molecular marker assisted breeding and breeding high-quality new sesame varieties.

What is claimed is:

1. A sesame high oleic acid gene, SiFAD2-1, characterized by its location on chromosome 4 of sesame and classified as an incomplete dominant control gene; this gene has a length of 3213 bp, and its nucleotide sequence is provided in SEQ ID No. 1.

2. The allele Sifad2-1 of the sesame high oleic acid gene SiFAD2-1, according to claim 1, characterized in that its nucleotide sequence is provided in SEQ ID No. 2.

3. A cDNA corresponding to the sesame high oleic acid gene SiFAD2-1 according to claim 1, characterized in that it differs from the cDNA corresponding to the allele Sifad2-1 of the sesame high oleic acid gene SiFAD2-1 is that the 1042^{nd} nucleotide is T;
the base sequence of the cDNA corresponding to the allele Sifad2-1 is provided in SEQ ID NO. 3.

4. A primer pair for obtaining the sesame high oleic acid gene SiFAD2-1 according to claim 1 or its allele Sifad2-1 according to claim 2 by PCR amplification, characterized in that the primer is designed as follows:
a positive Primer HO1 Primer F:
5′-TATGAGACCTCGTAAGGCAG-3′;
a reverse primer HO1 Primer R:
5′-GTAGGATGGAAGAAAATCTC-3′.

5. A PCR amplification method for obtaining the sesame high oleic acid gene SiFAD2-1 according to claim 1 or the allele Sifad2-1 according to claim 2, using the primer pair according to claim 4, characterized by comprising the following steps:
(1) extracting genomic DNA from a high oleic acid germplasm HO995 or a sesame sample with normal oleic acid content Yuzhi 11;
(2) using the genomic DNA extracted in step (1) as a template, performing PCR amplification with the primer pair;
when using the genomic DNA of the high oleic acid germplasm HO995 as the template, the sesame high oleic acid gene SiFAD2-1 is obtained after amplification;
when using the genomic DNA of the normal oleic acid content sesame sample Yuzhi 11 as the template, the allele Sifad2-1 is obtained after amplification.

6. A Primer pair for a HRM PCR detection of the sesame high oleic acid gene SiFAD2-1 according to claim 1 or its allele Sifad2-1, according to claim 2, wherein the primer pair is named SNP marker SiSNPfad2-1, and is designed as follows:
HSHO1-F sequence:
5′-AAGGGTTGGCTTGGCTGGTA-3′;
HSHO1-R sequence:
5′-CGGCAACGAAGGGTGAGTG-3′;
an amplification product has a length of 104 bp.

7. A method for detecting and determining a phenotype of sesame oleic acid content using the HRM PCR detection primer pair according to claim 6, comprising the following steps:
(1) extract genomic DNA from the sesame sample to be tested;
(2) perform a HRM PCR amplification using the genomic DNA extracted in step (1) as the template and the primer pair designed for HRM PCR detection;
(3) compare the peak result of the HRM PCR amplification with a control to determine whether the sample is a high oleic acid gene phenotype or a normal oleic acid gene phenotype.
when determining the genotype of the sample:
if a peak line is observed, the sample is identified a C/T phenotype and is classified as heterozygous;
if a smooth line is observed, determine whether the sample is a homozygous high oleic acid TT phenotype or a homozygous normal CC phenotype. In order to further distinguish the homozygous CC and TT phenotypes, an equal amount of normal oleic acid content control CC DNA template is added to the DNA of the to-be-identified sample, and a second HRM PCR is performed:
if a peak line appears in the amplification result, the sample is identified as TT phenotype, indicating the presence of the SNP mutation, and is determined to be a homozygous high oleic acid TT phenotype;
if the amplification result remains a smooth line, it indicates that the mixed sample is a homozygous CC phenotype, meaning the sample does not contain the T mutation SNP site and is determined to be a homozygous CC phenotype with normal oleic acid content.
